(19) United States
(12) Patent Application Publication
(10) Pub. No.: US 2014/0172120 A1
(43) Pub. Date: Jun. 19, 2014

(54) METHOD AND SYSTEM FOR A PROSTHETIC DEVICE WITH MULTIPLE LEVELS OF FUNCTIONALITY ENABLED THROUGH MULTIPLE CONTROL SYSTEMS

(71) Applicant: Freedom Innovations, LLC, Irvine, CA (US)

(72) Inventors: Wilson Steele, Huntington Beach, CA (US); Hugo Quintero, Santa Ana, CA (US); Paul Van Wieren, Costa Mesa, CA (US); Matthew P. McFadden, Ladera Ranch, CA (US)

(73) Assignee: Freedom Innovations, LLC, Irvine, CA (US)

(21) Appl. No.: 14/020,758

(22) Filed: Sep. 6, 2013

Related U.S. Application Data
(60) Provisional application No. 61/697,747, filed on Sep. 6, 2012.

(51) Int. Cl. A61F 2/68; A61F 2/64

(52) U.S. Cl. CPC A61F 2/68; A61F 2/64
USPC 623/39

(57) ABSTRACT

Systems and methods for a prosthetic device with multiple levels of functionality enabled through multiple control systems. The microprocessor-controlled prosthetic device stores a plurality of programmable control systems, such as a beginner control system, an intermediate control system, and an advanced control system. Based on a medical professional's assessment, the prosthetic device is programmed to operate under a programmable control system suitable to the capability level of the amputee. The microprocessor-controlled prosthetic device can be re-programmed to operate under another programmable control system as the capability level of the amputee changes. A new amputee should operate under a beginner control system, which provides high stability and low functionality. The high stability, low functionality combination provides high predictability, which helps the new amputee build confidence and trust in the prosthetic. As the amputee's capability progresses, increased functionality is provided in the intermediate and advanced control systems.

### CLAIM OF PRIORITY UNDER 35 U.S.C. §119(e)

This application claims the benefit and priority of U.S. Provisional Patent Application No. 61/697,747, entitled “SELECTABLE LEVELS OF FUNCTIONALITY FOR PROSTHETIC DEVICE,” filed on Sep. 6, 2012, the entire contents and disclosures of which are hereby incorporated by reference herein.

### BACKGROUND

1. Field of the Invention

The present invention relates generally to prosthetics, and in particular, methods and systems for a prosthetic device with multiple levels of functionality enabled through multiple control systems.

Typically, when a person first becomes an amputee, he is fitted with a preliminary prosthesis for purposes of acclimation. This acclimation period may last up to six months or more. The acclimation period usually includes physical therapy designed to train the new amputee to use and live with a prosthesis (or prostheses if multiple limbs were lost). The preliminary prosthesis is usually a temporary prosthesis with high stability but low functionality.

For a new amputee to be successful in learning to live with the prosthesis, it is critical that he learns to build confidence and trust in the prosthesis early on. In particular, the feeling of predictability contributes greatly to a new amputee's confidence and trust in the prosthesis. Hence, the temporary prosthesis's high stability feature is ideal for a new amputee because it leads to high predictability. The high stability feature reduces the likelihood of falling and thus, helps a new amputee build confidence in the prosthesis early on. Similarly, the temporary prosthesis's low functionality is ideal for a new amputee because there is higher predictability with low functionality. Otherwise, if a new amputee is given increased functionality early on, his learning and trust in the prosthesis will be hindered as the predictability and stability decreases. Therefore, increased functionality is detrimental to a new amputee's learning process.

After the acclimation period, the preliminary prosthesis is removed, and a definitive prosthesis is fitted. The definitive prosthesis is a more permanent prosthesis with increased functionalities and can be used for many years or the remaining lifetime of the amputee. Like the preliminary prosthesis, the amputee will likely need an acclimation period to learn to use and live with the definitive prosthesis and in particular, learn to use the increased functionalities. Thus, the switch from the preliminary prosthesis to the definitive prosthesis requires additional cost, time, and effort. Such additional cost, time, and effort are compounded if the amputee requires additional prostheses (i.e. lost multiple limbs).

In order to realize the increased functionalities associated with the definitive prosthesis, the definitive prosthesis requires a load bearing limb and proper control of the remnant musculature. It is important to note that a new amputee is usually not suitable for a prosthesis with increased functionalities because (a) the new amputee must first be properly trained to use the increased functionalities, and (b) the remnant limb may require time to heal rendering it incapable of serving as a load bearing limb required for the increased functionalities.

2. Description of the Related Art

Today, microprocessor-controlled prosthetics capable of mimicking the functionalities of a joint, such as a knee, are available in the market. Examples of such microprocessor-operated prostheses are described in U.S. Pat. No. 7,655,050, entitled “Computer controlled prosthetic knee device,” and U.S. patent application Ser. No. 13/015,414, entitled, “Novel enhanced methods for mimicking human gait with prosthetic knee devices,” the contents of which are hereby incorporated by reference.

Additionally, microprocessor-controlled prosthetic technology has progressed to the point that, high-speed, proportionally controlled valves with low enough power consumption to be battery operated, are available. Such valves can control fluid flow in a prosthetic device, such as a knee as demonstrated in U.S. patent application Ser. No. 13/829,714, entitled “Prosthetic with Voice Coil Valve.” With such valves and accompanying an hydraulic system, a single prosthetic device emulating a knee can be programmed to emulate the functionality of any other, pre-existing prosthetic device, again such as a knee, with only software or algorithm changes.

Moreover, current microprocessor-operated prostheses allow the amputee himself to switch between modes at his convenience by, for example, simply pressing the appropriate control button. For example, the amputee can switch from a driving mode to riding-a-bike mode. However, such prostheses and accompanying functionalities are not appropriate for a new amputee. Thus, such prostheses still suffers from the limitation of requiring a temporary prosthesis before a more definitive prosthesis.

In view of the foregoing, there is a need for a new amputee to be fitted initially with a single prosthesis that has functionalities associated with a preliminary prosthesis and a definitive prosthesis such that a separate preliminary prosthesis can be bypassed entirely. In doing so, the switch from the preliminary prosthesis to the definitive prosthesis is no longer required, resulting in reduced cost, time, and effort. Additional reductions in cost, time and effort are realized if the amputee requires multiple prostheses (i.e. lost multiple limbs).

The present invention fulfills the foregoing needs by introducing a more sophisticated, safer, and effective system and method for training a new amputee to live with a prosthesis (or prostheses if multiple limbs are lost) without the need of a preliminary prosthesis. The present invention's prosthetic joint essentially serves as both the preliminary prosthesis and the definitive prosthesis. In particular, the present invention introduces methods and systems for a prosthetic device with multiple levels of functionality enabled through multiple control systems, wherein each control system provides for different functional capabilities matched to the capability of the amputee as he progresses from a new user to an advanced user. The term ‘user’ and the term ‘amputee’ are used interchangeably herein. At the minimum, the present invention discloses at least three control systems: (1) beginner (i.e. a new amputee learning to stand, sit, and walk slowly), (2) intermediate (i.e. normal walking), and (3) advanced (i.e. sporting-related activities). Such control systems can be altered with changes to the software or algorithm.

The core concept of the present invention is the ability of a single prosthetic device to be configured for one of a plurality of control systems, the purpose of which is to have a single device capable of drastically different functionality. This could be used to match the progression of an amputee who is changing from a new user to a more advanced user. The idea is to match the functionality of the prosthesis with the capability of the user, which is likely to change over time.

### SUMMARY OF THE INVENTION

The present invention fulfills the foregoing needs by introducing methods and systems for a prosthetic device with multiple levels of functionality enabled through multiple control systems. By including multiple control systems within a single prosthesis device, a new amputee can be fitted with a definitive prosthesis early on, and thus reap the benefits of reduced costs, time, and energy. The control systems are programmable by software or algorithm. The present invention discloses at least three programmable control systems: (1) beginner (i.e. a new amputee learning to stand, sit, and walk slowly), (2) intermediate (i.e. normal walking), and (3) advanced (i.e. sporting-related activities). As the amputee progresses from the beginner control system to the advanced control system, his capability level should increase and thus, the functionalities available to the amputee should increase accordingly.

The multiple programmable control systems distinguish the present invention from the multiple modes of the related art. In particular, the present invention's programmable control systems are essentially different states of a program that an amputee cannot unilaterally and freely access in and out of. The multiple programmable control systems are mutually exclusive such that the prosthetic device can only operate under one control system at any given time. It is contemplated that a qualified medical professional, such as a prosthetist, determines the appropriate control system for the amputee and sets the prosthesis accordingly. Once a control system is set, the prosthetic device can only operate the set of functionalities available under the selected control system.

Additionally, a control system is not as limiting as a mode because while a control system may include multiple modes, a mode cannot include multiple control systems. For example, the present invention's advanced control system can encompass many different modes, such as a running mode, a driving mode, a biking mode, and a golfing mode. Furthermore, unlike the multiple modes of the related art, the present invention's programmable control systems can be readily changed by software or algorithm changes. In sum, the present invention introduces a more sophisticated, safer, and effective system and method for training new amputees on how to use microprocessor-operated prosthesis (or multiple prostheses if necessary).

An exemplary embodiment of the present invention's system for a prosthetic device with multiple levels of functionality enabled through multiple control systems comprises a master control unit configured to program a microprocessor-controlled prosthetic device; the microprocessor-controlled prosthetic device having a controller, an actuator, a plurality of sensors, and a machine-readable storage medium; the controller having a microprocessor; the machine-readable storage medium for storing a plurality of programmable control systems, wherein each of the plurality of programmable control systems has at least one functionality different from each other; and an interface configured to receive an external input, wherein the controller is configured to receive the external input from the interface.

Another exemplary embodiment of the present invention's system for a prosthetic device with multiple levels of functionality enabled through multiple control systems comprises a master control unit configured to program a microprocessor-controlled prosthetic device; the microprocessor-controlled prosthetic device having a controller, an actuator, a plurality of sensors, and a machine-readable storage medium; the controller having a microprocessor; the machine-readable storage medium for storing a plurality of programmable control systems, wherein each of the plurality of programmable control systems has at least one functionality different from each other, and wherein the plurality of programmable control systems includes at least a beginner control system, an intermediate control system, and an advanced control system; and an interface configured to receive an external input, wherein the controller is configured to receive the external input from the interface.

Another exemplary embodiment of the present invention's system for a prosthetic device with multiple levels of functionality enabled through multiple control systems comprises a master control unit configured to program a microprocessor-controlled prosthetic device; the microprocessor-controlled prosthetic device having a controller, an actuator, a plurality of sensors, and a machine-readable storage medium; the controller having a microprocessor; the machine-readable storage medium for storing a plurality of programmable control systems, wherein each of the plurality of programmable control systems has at least one functionality different from each other, wherein the plurality of programmable control systems includes at least a beginner control system, an intermediate control system, and an advanced control system, and wherein the beginner control system is a weight-activated stance control system, the intermediate control system is a swing-and-stance control system, and the advanced control system is an activities of daily living control system; and an interface configured to receive an external input, wherein the micro-controller is configured to receive the external input from the interface.

Another exemplary embodiment of the present invention's system for a prosthetic device with multiple levels of functionality enabled through multiple control systems comprises a master control unit configured to program a microprocessor-controlled prosthetic device; the microprocessor-controlled prosthetic device having a controller, an actuator, a plurality of sensors, and a machine-readable storage medium; the actuator having a hydraulic damping unit, wherein the hydraulic damping unit has a computer-controlled proportional valve, a spool valve, a reed check valve, and a return spring; the controller having a microprocessor; the machine-readable storage medium for storing a plurality of programmable control systems, wherein each of the plurality of programmable control systems has at least one functionality different from each other; and an interface configured to receive an external input, wherein the controller is configured to receive the external input from the interface.

An exemplary embodiment of the present invention's prosthetic device with multiple levels of functionality enabled through multiple control systems comprises a controller having a microprocessor; a plurality of sensors; an actuator; a machine-readable storage medium for storing a plurality of programmable control systems, wherein each of the plurality of programmable control systems has at least one functionality different from each other; and an interface configured to receive an external input, wherein the controller is configured to receive the external input from the interface.

Another exemplary embodiment of the present invention's prosthetic device with multiple levels of functionality enabled through multiple control systems comprises a controller having a microprocessor; a plurality of sensors; an actuator; a machine-readable storage medium for storing a plurality of programmable control systems, wherein each of the plurality of programmable control systems has at least one functionality different from each other, and wherein the plurality of programmable control systems includes at least a beginner control system, an intermediate control system, and an advanced control system; and an interface configured to receive an external input, wherein the controller is configured to receive the external input from the interface

Another exemplary embodiment of the present invention's prosthetic device with multiple levels of functionality enabled through multiple control systems comprises a controller having a microprocessor; a plurality of sensors; an actuator; a machine-readable storage medium for storing a plurality of programmable control systems, wherein each of the plurality of programmable control systems has at least one functionality different from each other, wherein the plurality of programmable control systems includes at least a beginner control system, an intermediate control system, and an advanced control system, and wherein the beginner control system is a weight-activated stance control system, the intermediate control system is a swing-and-stance control system, and the advanced control system is an activities of daily living control system; and an interface configured to receive an external input, wherein the controller is configured to receive the external input from the interface.

Another exemplary embodiment of the present invention's prosthetic device with multiple levels of functionality enabled through multiple control systems comprises a controller having a microprocessor; a plurality of sensors; an actuator; the actuator having a hydraulic damping unit, wherein the hydraulic damping unit has a computer-controlled proportional valve, a spool valve, a reed check valve, and a return spring; a machine-readable storage medium for storing a plurality of programmable control systems, wherein each of the plurality of programmable control systems has at least one functionality different from each other; and an interface configured to receive an external input, wherein the controller is configured to receive the external input from the interface.

An exemplary embodiment of the present invention's method for a prosthetic device with multiple levels of functionality enabled through multiple control systems comprises the steps of connecting the microprocessor-controlled prosthetic device to an external input; assessing a capability level of the amputee; selecting a programmable control system from a plurality of programmable control systems that is suitable for the amputee based on the capability level of the amputee, wherein each of the plurality of programmable control systems has at least one functionality different from each other; programming the microprocessor-controlled prosthetic device to operate the selected programmable control system, wherein the external input programs the microprocessor-controlled prosthetic device; and disconnecting the microprocessor-controlled prosthetic device from the external input.

Another exemplary embodiment of the present invention's method for a prosthetic device with multiple levels of functionality enabled through multiple control systems comprises the steps of connecting the microprocessor-controlled prosthetic device to an external input; assessing a capability level of the amputee; selecting a programmable control system from a plurality of programmable control systems that is suitable for the amputee based on the capability level of the amputee, wherein each of the plurality of programmable control systems has at least one functionality different from each other, and wherein the plurality of programmable control systems includes at least a beginner control system, an intermediate control system, and an advanced control system; programming the microprocessor-controlled prosthetic device to operate the selected programmable control system, wherein the external input programs the microprocessor-controlled prosthetic device; and disconnecting the microprocessor-controlled prosthetic device from the external input.

Another exemplary embodiment of the present invention's method for a prosthetic device with multiple levels of functionality enabled through multiple control systems comprises the steps of connecting the microprocessor-controlled prosthetic device to an external input; assessing a capability level of the amputee; selecting a programmable control system from a plurality of programmable control systems that is suitable for the amputee based on the capability level of the amputee, wherein each of the plurality of programmable control systems has at least one functionality different from each other, wherein the plurality of programmable control systems includes at least a beginner control system, an intermediate control system, and an advanced control system, and wherein the beginner control system is a weight-activated stance control system, the intermediate control system is a swing-and-stance control system, and the advanced control system is an activities of daily living control system; programming the microprocessor-controlled prosthetic device to operate the selected programmable control system, wherein the external input programs the microprocessor-controlled prosthetic device; and disconnecting the microprocessor-controlled prosthetic device from the external input.

Another exemplary embodiment of the present invention's method for a prosthetic device with multiple levels of functionality enabled through multiple control systems comprises the steps of connecting the microprocessor-controlled prosthetic device to an external input; a qualified medical professional assessing a capability level of the amputee; the qualified medical professional selecting a programmable control system from a plurality of programmable control systems that is suitable for the amputee based on the capability level of the amputee, wherein each of the plurality of programmable control systems has at least one functionality different from each other; programming the microprocessor-controlled prosthetic device to operate the selected programmable control system, wherein the external input programs the microprocessor-controlled prosthetic device; and disconnecting the microprocessor-controlled prosthetic device from the external input.

The contents of this summary section are provided only as a simplified introduction to the invention, and are not intended to be used to limit the scope of the appended claims. The present disclosure has been described above in terms of presently preferred embodiments so that an understanding of the present disclosure can be conveyed. However, there are other embodiments not specifically described herein for which the present disclosure is applicable. Therefore, the present disclosure should not to be seen as limited to the forms shown, which is to be considered illustrative rather than restrictive.

### BRIEF DESCRIPTION OF THE DRAWINGS

Other systems, methods, features and advantages of the present invention will be or will become apparent to one of ordinary skill in the art upon examination of the following figures and detailed descriptions. It is intended that all such additional apparatuses, systems, methods, features and advantages be included within this description, be within the scope of the present invention, and be protected by the appended claims. Component parts shown in the drawings are not necessarily to scale, and may be exaggerated to better illustrate the important features of the present invention. In the drawings, like reference numerals designate like parts throughout the different views, wherein:

FIG. 1 is a box diagram of an exemplary embodiment of the present invention's system for a prosthetic device with multiple levels of functionality enabled through multiple control systems.

FIG. 2 depicts an exemplary gait cycle and corresponding states of a prosthetic joint according to an embodiment of the present invention.

FIG. 3 is a box diagram depicting the present invention's programmable control systems and exemplary embodiments of each control system.

FIG. 4 is a flowchart depicting an exemplary embodiment of the present invention's method for a prosthetic device with multiple levels of functionality enabled through multiple control systems.

FIG. 5 is a representative data plot of sensor streams from an exemplary prosthetic knee, and included in the plot are unit state, knee angle, knee angular velocity, moment, load, pressure A, and pressure B.

FIG. 6 is a descriptive algorithm path of a weight-activated stance control system.

FIG. 7 is a descriptive data stream of a weight-activated stance control system.

FIG. 8 is a descriptive algorithm path of a multiple gait speed, swing-and-stance control system.

FIG. 9 is a descriptive data steam of a multiple gait speed, swing-and-stance control system.

FIG. 10 is a descriptive algorithm path of an activities of daily living (ADL) control system.

FIG. 11 is a descriptive data stream path of an ADL control system for the level ground walking loop.

### DETAILED DESCRIPTION

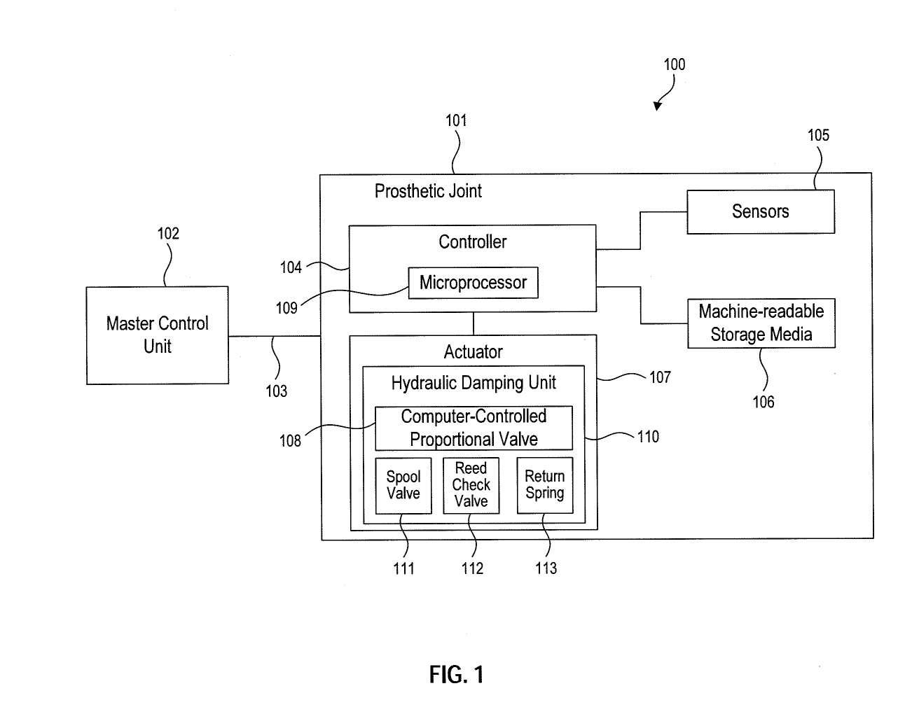

FIG. 1 is a box diagram of an exemplary embodiment of the present invention's system for a prosthetic device with multiple levels of functionality enabled through multiple control systems. Prosthetic joint control system 100 has prosthetic joint 101 and master control unit 102. Prosthetic joint 101 has controller 104, machine-readable storage media 106, a plurality of sensors 105, and an actuator 107.

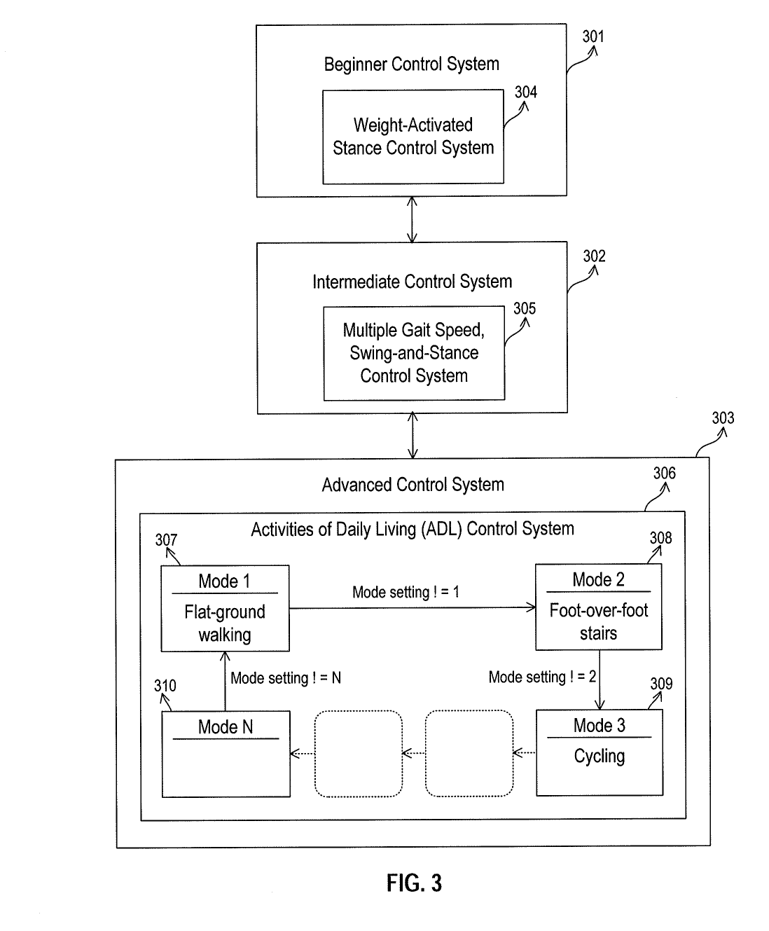

Prosthetic joint 101 can communicate with master control unit 102 via communication media 103. Communication media 103 typically embodies machine-readable instructions, data structures, program modules, or other data in a modulated data signal such as a carrier wave or other transport mechanism and includes any information delivery media. By way of example, and not limitation, communication media 103 includes wired media such as a wired network or direct-wired connection, and wireless media such as acoustic, RF, infrared, or other wireless media. In the present invention, it is contemplated that a qualified medical professional, such as a prosthetist, operates and controls master control unit 102. Thus, the qualified medical professional makes the decision to communicate and transfer data with prosthetic joint 101. Such data provides the instructions as to which control system, as shown in FIG. 3, shall operate prosthetic joint 101.

Controller 104 serves as the central processing unit of prosthetic joint 101. Controller 104 has microprocessor 109. Microprocessor 109 is any microprocessor known to one skilled in the art, such as an Intel microprocessor, an AMD microprocessor, and the like. As known to one skilled in the art, microprocessor 109 is typically mounted on a printed circuit board (PCB), which allows microprocessor 109 to communicate with machine-readable storage media 106, sensors 105, and actuator 107.

Machine-readable storage media 106 stores a plurality of programmable control systems, and other instructions or data which implement all or part of the invention described herein. The programmable control systems are computer-executable instructions for operating prosthetic joint 101. These programmable control systems are transferred from master control unit 102 to machine-readable storage media 106. As shown in FIG. 3, the present invention includes at least three programmable control systems: (1) beginner control system 301 (i.e. a weight-activated control system 304 for a new amputee), (2) intermediate control system 302 (i.e. multiple gait speed, swing-and-stance control system 305 to achieve normal gait walking), and (3) advanced control system 303 (i.e. an activities of daily living control system 306 for sporting-related activities). Master control unit 102 instructs controller 104 to select the desired control system from machine-readable storage media 106. Upon selection of the desired control system, prosthetic joint 101 can only perform the functionalities provided by the selected control system. The control systems are mutually exclusive such that prosthetic device 101 only operates under the selected control system during the time period in which such system is selected.

Machine-readable storage media 106 can be any available media that can be accessed by controller 104, removable and non-removable media implemented by any method or technology for storage of information such as computer-readable instructions, data structures, program modules, or other data. Machine-readable storage media includes, but is not limited to, RAM, ROM, EEPROM, flash memory, portable memory, or other memory technology, CD-ROM, digital versatile disks (DVD), or other optical disk storage, magnetic cassettes, magnetic tape, magnetic disk storage or other magnetic storage devices, or any other medium which can be used to store the desired information and which can be accessed by controller 104, including remote storage accessed over a wireless connection, such as Bluetooth or 402.11-type wireless communication signals.

Sensors 105 detect and collect data of prosthetic joint movement. Such data is transferred to controller 104. Sensors 105 are positioned throughout prosthetic joint 101. The positioning of sensors 105 can vary depending on design needs and the type of joint. Controller 104 can determine when the transition from the stance phase to the swing phase of the gait cycle should occur and actuate spool valve 111 of the hydraulic damping unit 110 at the proper time.

Multiple different types of sensing are required to enable an exemplary passive prosthetic device to function in the modern world, sense the desired motion or stiffness required by the amputee, and react accordingly with the correct time frame. For an exemplary prosthetic device, such as a knee, these sensed signals could include a time count, knee angle, knee angular velocity, internal hydraulic pressure, force applied, and bending torque applied, gyroscopic and acceleration forces from the world frame,

Weight is viewed through at least two sensors, the load sensor and moment sensor. The load sensor the force applied by the person to the distal end of the device, much like a bathroom scale senses force applied. The moment sensor is able to discern the bending torque applied, such as the forces applied by a torque wrench one might use to tighten lug nuts on a car. Both the load and moment sensor, as combined in a single unit, are described in U.S. patent application Ser. No. 13/015,423, entitled “Compact and robust load and moment sensor.” Other embodiments of the sensor, either combined, or separate, are obvious to one skilled in the art. For example, both described inputs above are sensed in the sagittal plane, and additional sensors could be utilized which sense in the frontal or transverse plane to allow the internal microcontroller greater visibility to the external world.

Knee angle, and knee angular velocity, could be seen by the device's microcontroller using from the methods laid out in U.S. patent application Ser. No. 13/015,442, entitled “Angle measurement device and method.” Other methods of robustly sensing an angle of a prosthetic device are obvious to one skilled in the art.

Actuator 107 has a hydraulic damping unit 110, which includes computer-controlled proportional valve 108, spool valve 111, reed check valve 112, and a return spring 113. Controller 104 controls computer-controlled proportional valve 108. When computer-controlled proportional valve 108 is energized, it opens spool valve 111 and permits fluid to flow through the spool valve 111.

Thus, hydraulic damping unit 110 and actuator 107 operate in a binary operation: stiff configuration (with increased damping and high resistance) to loose, freely swinging configuration (with decreased damping and low resistance) depending on what portion of the gait cycle the amputee is in. Controller 104 controls energy flow to computer-controlled proportional valve 108 based on prosthetic joint movement data detected by sensors 105 and prosthetic joint movement decision values stored in machine-readable storage media 106.

In this exemplary embodiment, where hydraulic fluids and chambers are utilized to allow the prosthetic device to react to the environment and create externally felt torque or force, pressure sensors could be used to sense pressure on both sides of the hydraulic working chamber. Small, in-expensive, and highly compact pressure sensors are commonly available on the market, and are exemplified by those from Measurement Specialties, Inc., such as their part number 86-BSD, a miniature digital pressure sensor.

There are a great number of examples of battery operated microelectronic systems on the market place today which use integrated circuit gyro and accelerometer sensing elements. Modern cellular phones and tablets use these chips to re-orient the screen as one changes the orientation of the device. These units have come down greatly in price, and are now easily included on printed circuit board designs.

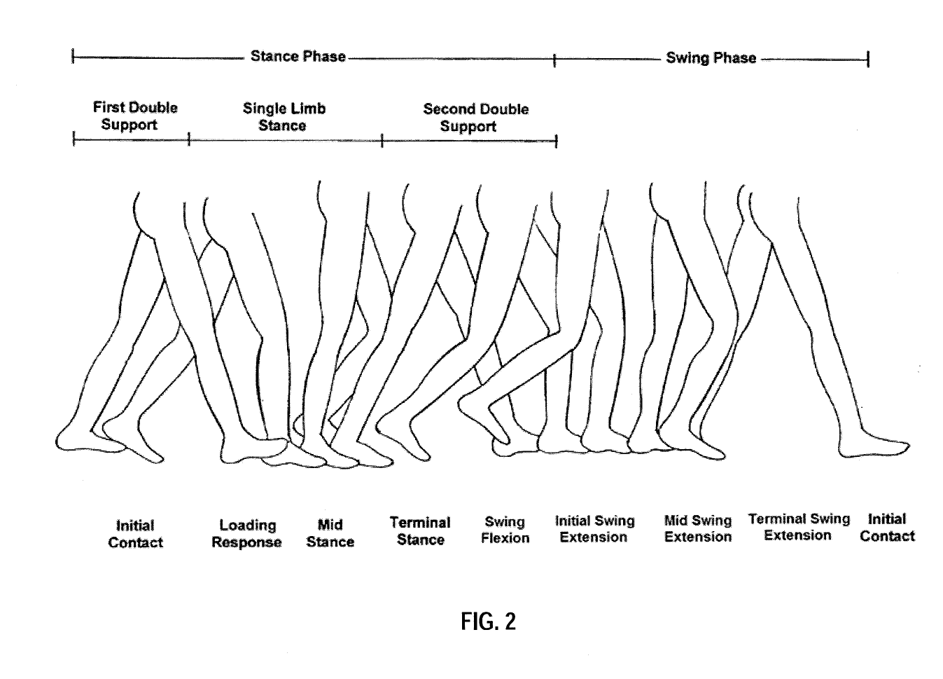

FIG. 2 depicts an exemplary gait cycle and corresponding states of a prosthetic joint according to an embodiment of the present invention. Makers of prosthetic knees have long attempted to mimic a natural walking gait. For purpose of illustration, this may be understood to be a reference to an exemplary walking gait cycle on level-ground, as is presented in FIG. 2. During intermediate control system 302, the amputee learns to mimic a natural walking gait.

A walking gait can be divided into two phases, a stance phase and a swing phase. The stance phase is defined as the period of time during which the foot of the observed leg is weighted. The swing phase is defined as the period of time when the foot of the observed leg is un-weighted. Within stance are multiple sub-phases such as the initial contact sub-phase, the loading response sub-phase, the mid stance sub-phase, the terminal stance sub-phase, the swing flexion sub-phase, the initial swing extension sub-phase, the mid-swing extension sub-phase, and/or the terminal swing extension sub-phase. The cycle repeats itself again on initial contact, or heel strike.

The stance phase of a walking gait begins as the heel strikes the ground, indicated by the sub-phase initial contact. Upon heel strike, the knee flexes slightly to absorb some of the impact forces acting on the limb due to weight acceptance as indicated by the loading response sub-phase.

After the foot is flat on the ground, the shin begins to rotate forward about the ankle as indicated by the mid stance sub-phase. During mid-stance, the shin rotates, and the knee remains flexed in order to minimize the rise of the person's center of mass as it passes over the ankle joint center. As the shin rotates forward and the center of mass progresses forward, the weight acting on the limb moves quickly towards the toe of the foot. The force of the weight acting on the toe generates a torque about the knee joint that tends to straighten, or extend, the knee—referred to as “stance extension” of the knee. Stance extension continues until the transition point to the swing phase, and is also known as geometric locking because of the ground reaction force acting to keep the knee straight and capable of load bearing.

Soon after the knee is completely extended, the toe pushes off the ground, as indicated by the terminal stance sub-phase. As the toe pushes off the ground, the knee begins to flex as indicated by the swing flexion sub-phase. The knee flexes to about 60° degrees in the swing flexion sub-phase. In order to keep the toe from stubbing on the ground, the knee will remain flexed as the leg swings, forward about the hip joint in the initial swing extension sub-phase. As the leg continues to swing forward the knee will begin to extend until it is nearly straight —referred to as “swing extension” of the knee. Soon after, the knee is fully extended in the terminal swing extension sub-phase. Thereafter, the heel of the foot will strike the ground again, and the gait cycle begins all over.

During the level-ground walking gait cycle described above, a biological knee, together with the muscles acting on it, functions primarily as an absorber of energy. Thus, to mimic a natural walking gait and to support the amputee, hydraulic damping unit 110 provides a relatively high amount of resistance to motion, or damping, making prosthetic joint 101 comparatively stiff and able to support high forces. However, at certain times, hydraulic damping unit 101 provides a relatively low resistance making prosthetic joint 101 comparatively loose and able to swing freely to facilitate the natural gait of the amputee. For example, prosthetic joint 101 should be loose during portions of the terminal stance sub-phase, the swing flexion sub-phase, and the initial swing sub-phase. However, even in those sub-phases, prosthetic joint 101 may become stiff when there is abnormal gait event, such as when the amputee is falling or stumbling.

Based on the movements of the amputee in the normal gait cycle, controller 104 controls whether prosthetic joint 101 should be stiff or loose to both facilitate the normal gait of the amputee and also to aid the amputee when the amputee is falling or has an abnormal gait. To do so, controller 104 controls which state prosthetic joint 101 is in. As seen in FIG. 2, prosthetic joint 101 is in generally one of the following states during a natural gait: Initial Contact, Loading Response, Mid Stance, Terminal Stance, Swing Flexion, Initial Swing Extension, Mid Swing Extension, Terminal Swing Extension, Initial Contact (repeat). Generally controller 104 starts prosthetic joint 101 at the Initial Contact state and progresses through the states unless there are any abnormalities, in which case controller 104 returns the state of prosthetic joint 101 to the Initial Contact state.

FIG. 3 is a box diagram depicting the present invention's programmable control systems and exemplary embodiments of each control system. The present invention introduces at least three programmable control systems: (1) beginner control system 301 (i.e. a new amputee learning to stand, sit, and walk slowly), (2) intermediate control system 302 (i.e. normal walking), and (3) advanced control system 303 (i.e. sporting-related activities). Tuning of these programmable control systems to the user can be accomplished with software or algorithm changes. As shown in FIG. 1, these programmable control systems can be programmed and modified by master control unit 102, then transferred to prosthetic joint 101 via communication media 103, and ultimately stored in machine-readable storage media 106. Controller 104 operates prosthetic joint 101 under the selected programmable control system. The programmable control systems are mutually exclusive such that prosthetic joint 101 only operates under the selected control system during the time period in which such system is selected.

Beginner control system 301 is characterized by high stability and low functionality. Beginner control system 301 is the default control system and is typically appropriate for a new amputee. Generally, a new amputee has a few basic aims: (a) get comfortable living with a prosthetic, (b) master the basic functionalities of a prosthetic, such as learning to stand without falling, and (c) building confidence and trust in the prosthetic. Beginner control system 301 is characterized by high stability, which leads to high predictability. High predictability helps the new amputee build confidence and trust in the prosthetic. The feeling of predictability contributes significantly to a new amputee's confidence and trust in the prosthetic. Thus, increased functionalities are not appropriate for a new amputee because it lowers predictability and therefore, increased functionalities would be detrimental to a new amputee's learning to live with, and build confidence and trust in, the prosthetic. Thus, beginner control system 301 has limited functionality for the amputee to sit, stand, and walk slowly with the prosthetic.

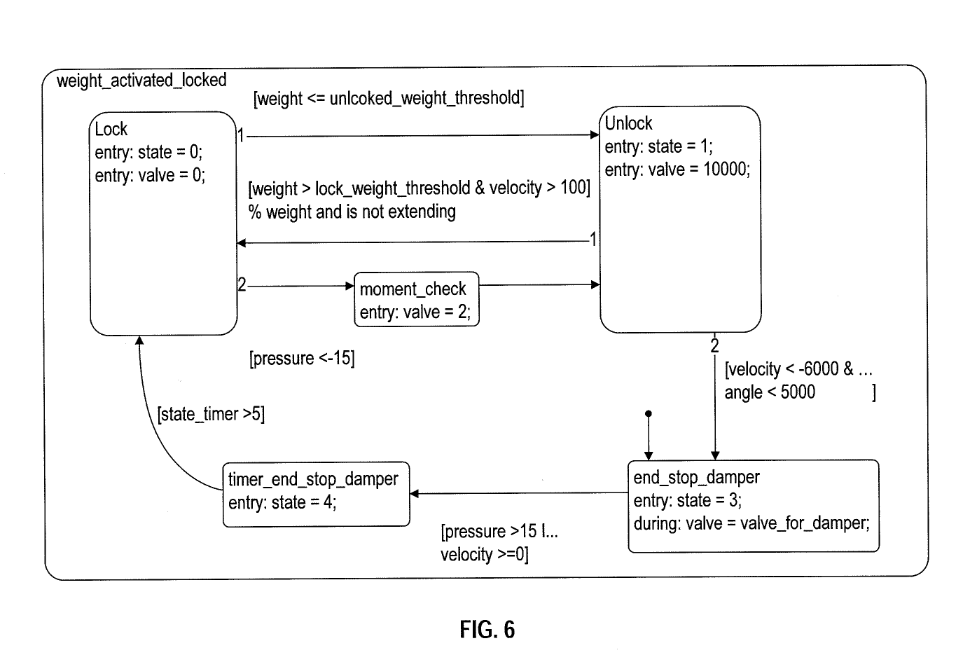

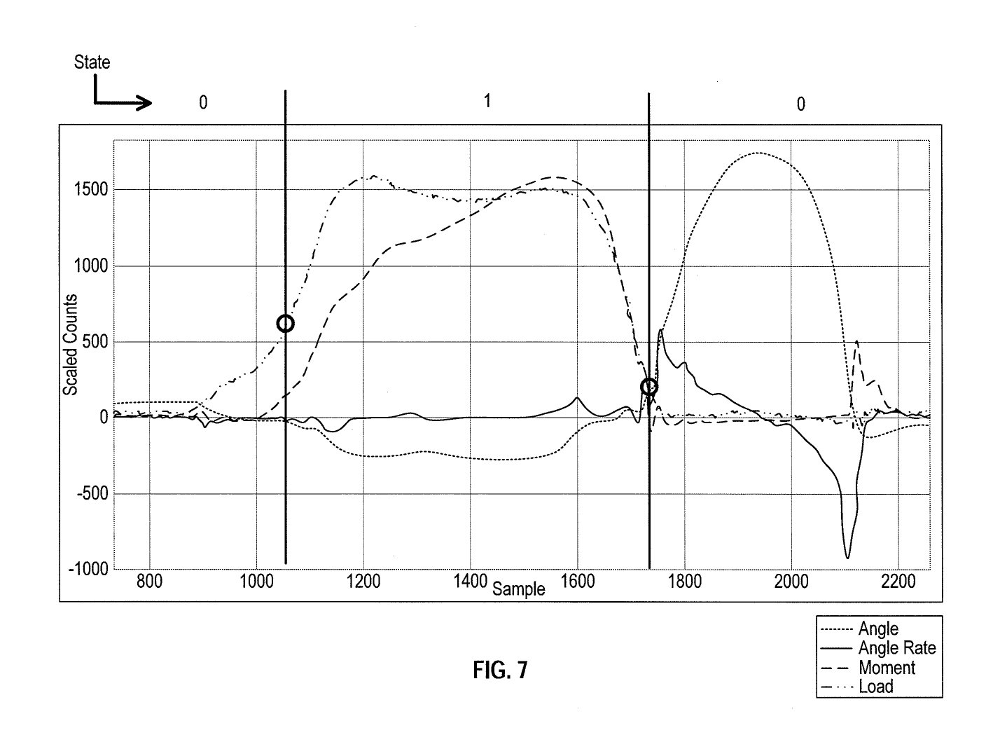

An exemplary embodiment of beginner control system 301 is weight-activated stance control system 304. In discussing weight-activated stance control system 304, reference is made to FIGS. 6-7, which show the descriptive algorithm path and descriptive data stream for weight-activated stance control system 304, respectively.

For a knee, weight-activated stance control system 304 is based on the principals of a weight activated stance type prosthetic knee, or a friction lock knee. The low cost units are typically fitted to an amputee who is just beginning rehab, is not a high capability or variable cadence walker, and is primarily looking for predictable safety and support from a knee unit, not smooth, natural, or low energy gait.

The basic tenant of weight-activated stance control system 304 is that the knee is completely stiff (or locked) when the amputee puts weight on it, and is essentially a hinge (unlocked state) when the amputee does not put weight on it.

Initial amputee weight is set by having prosthetic joint 101 support the whole body mass at an initial fitting to generate a [load] signal, and then taking one fully loaded step to generate a [moment] signal. These two signals, [load] and [moment] and then combined into a derived channel signal named [weight]. This single parameter greatly lowers the burden of setup on the prosthetist, and ensures that the amputee can gain basic functionality from prosthetic joint 101 with no learning burden.

As the load and moment sensor inputs are combined into a single [weight] variable, and a threshold (% of amputee body weight as) is set from the factory at 15%, there is no setup required for the device, by either the prosthetist or the amputee.

As well, the [velocity] signal is used within the loop of weight-activated stance control system 304 to enable prosthetic joint 101 to go to a stiff (locked) mode when prosthetic joint 101 is flexing with a greater rate than could conceivably be employed by an amputee of this skill level. This variable, [velocity], is simply the time based differential of the [knee angle] variable, which comes from an angle sensor within prosthetic joint 101.

In addition, a timer is employed such that an amputee cannot have prosthetic joint 101 in the free-swinging or “unlocked” mode for more than some set length of time, with a default setting of 5 seconds.

Intermediate control system 302 is characterized by intermediate stability and intermediate functionality. Intermediate control system 302 is for an amputee that has mastered beginner control system 301, or an amputee not yet suitable for advanced control system 303. A medical professional, such as a prosthetist, assesses the capability level of the amputee and determines whether intermediate control system 302 is appropriate for the amputee. Intermediate control system 302 presents increased functionalities not present in beginner control system 301. Intermediate control system 302 presents increased functionalities that allow the amputee to use the knee more efficiently and comfortably.

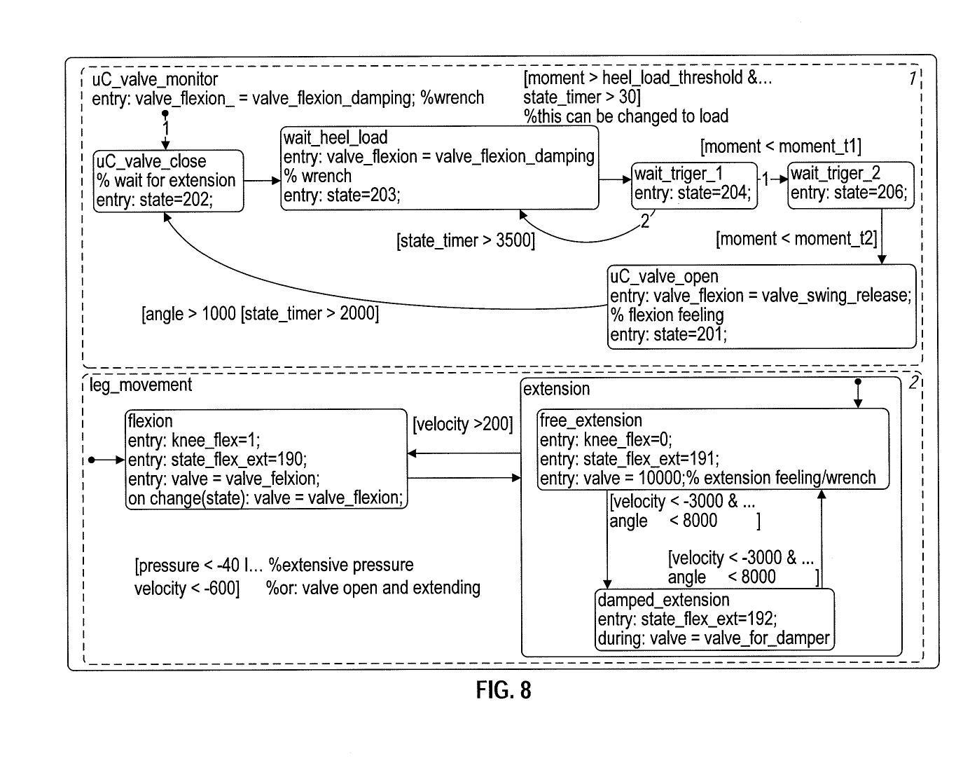

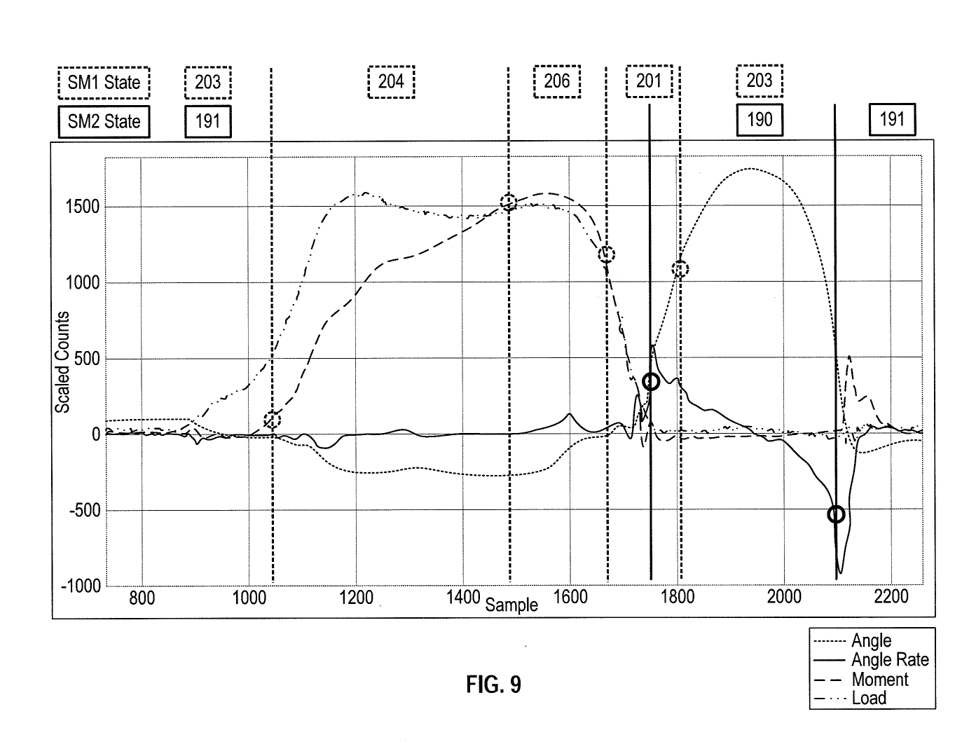

An exemplary embodiment of intermediate control system 302 is multiple gait speed, swing-and-stance control system 305. In discussing multiple gait speed, swing-and-stance control system 305, reference is made to FIGS. 8-9, which show the descriptive algorithm path and descriptive data stream for multiple gait speed, swing-and-stance control system 305, respectively.

Multiple gait speed, swing-and-stance control system 305 enables several different components of level ground walking in swing and in stance. Multiple gait speed, swing-and-stance control system 305 consists of two basic state machines that run simultaneously.

Prosthetic joint 101 enters the state diagram with the valve closed, and is waiting to see heel load by looking at the [moment] variable, specifically [moment] less than zero. A timer [state_timer] watches that the amputee hasn't stayed in this state too long, and backs them up a state if necessary. After this phase, the [moment] variable is tracked to see loading above the user set variable, [moment_{—}1]. After [moment_{—}1] is achieved, the [moment] variable is tracked to determine if it is below the user set variable, [moment_{—}2]. When the [moment] from live data is below the [moment_{—}2] variable, the valve is opened to allow swing flexion for that step. Once an appropriate value of swing flexion has been achieved as measured against the variable [angle], which is set to 65 deg+/−15%, the first state machine commands the valve to close. As well, if the valve has stayed open for too long, as referenced against [state_timer], the first state machine will also command the valve to close.

However, as there are two state machines active within multiple gait speed, swing-and-stance control system 305, when the first state machine commands the valve to close, if the second state machine sees that the amputee is extending the leg, it will over-ride the valve closing command until the amputee is done extending the leg. At all patterns of gait, if the amputee is trying to extend a knee device (i.e. prosthetic joint 101), the computer (i.e. controller 104) should manage the hydraulics to allow this action, and open the valve accordingly.

As an amputee walks with a prosthetic device, and in this case a knee (i.e. prosthetic joint 101), the amputee does not want the knee to come rapidly to full extension (in the terminal swing extension phase) and bang against the end stops. In order to appropriately slow the angular velocity of prosthetic joint 101 when the leg is almost at full extension, such as at 8 or 10 degrees from full extension, the microcontroller will command small amounts of valve closure to meter the hydraulic flow and slowly bring the knee to a halt, before heel strike, at initial contact. If the leg is extending, the second state diagram is usually in {free_extension}. However, if the {damped extension} sub state determines where the leg is in travel by looking at [angle] and [velocity], and sees that the leg is almost extended, it will command slight valve closure based on set points.

Multiple gait speed, swing-and-stance control system 305 is partially based on the invention disclosed in U.S. Pat. No. 8,444,704, entitled “Enhanced methods for mimicking human gait with prosthetic knee devices,” and U.S. patent application Ser. No. 13/015,414, entitled “Novel enhanced methods for mimicking human gait with prosthetic knee devices,” bearing in mind that those two disclosures incorporate significant functionality with the hydraulics of the disclosed system. The use of advanced hydraulic systems to react at the speed of sound to amputee inputs, which are external to computer control, by definition would dis-allow several tenants of the system described within this document. In order for a microprocessor controlled prosthetic joint, such as a knee (i.e. prosthetic joint 101), to emulate the functionality of other device, such as knees, with only software or control system changes, the entirety of the functionality of the device must be under the control of that software.

Advanced control system 303 is characterized by high functionality and low stability. Advanced control system 303 is for an amputee that has mastered control system 302 and thus, is typically appropriate for a fully recovered, experienced amputee. A medical professional, such as a prosthetist, assesses the capability level of the amputee and determines whether advanced control system 303 is suitable for the amputee.

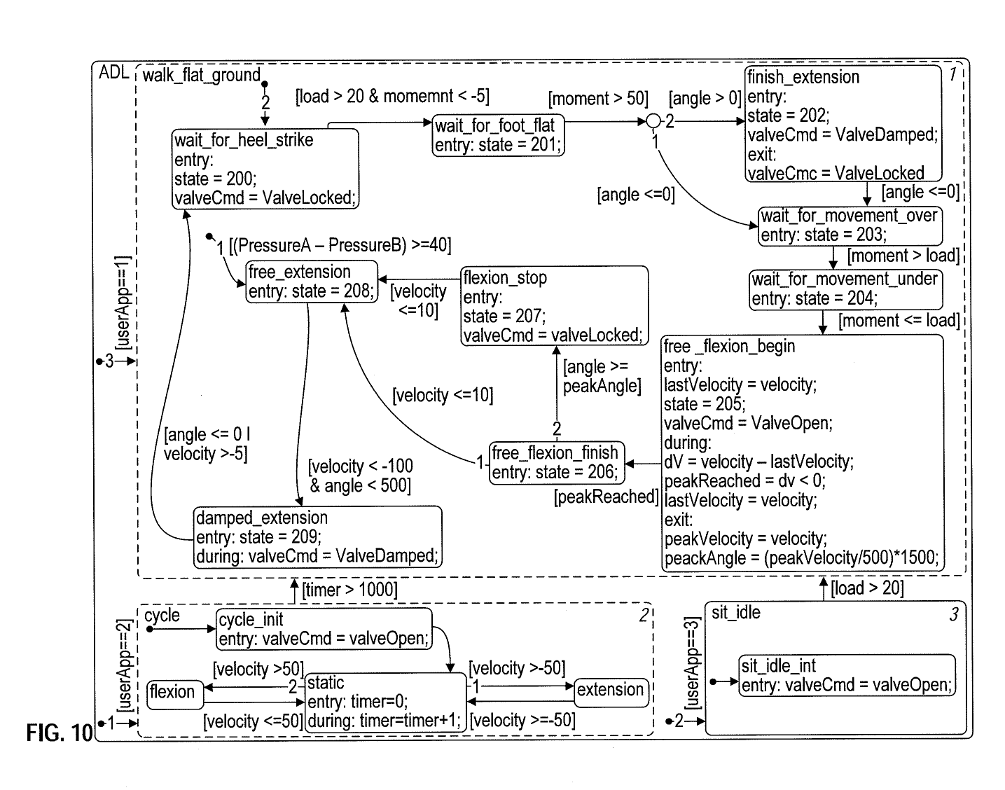

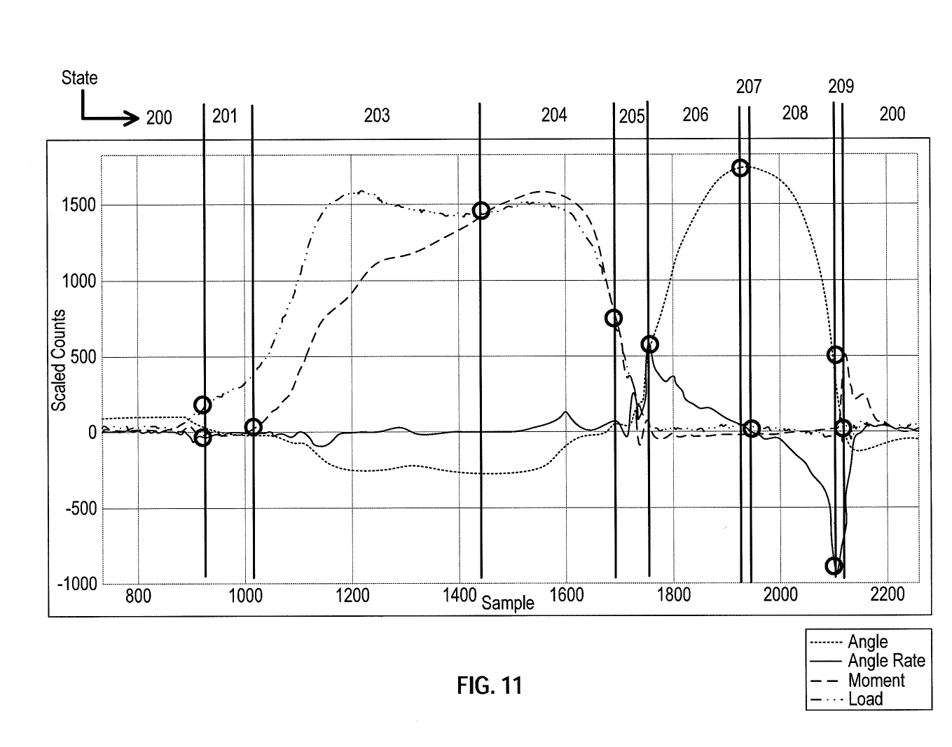

An exemplary embodiment of advanced control system 303 is activities of daily living (ADL) control system 306. In discussing ADL control system 306, reference is made to FIGS. 10-11, which show the descriptive algorithm path and descriptive data stream for ADL control system 306, respectively.

ADL control system 306 is intended to be a comprehensive control system state machine to enable all activities of daily living. ADL control system 306 also enables the amputee to utilize special modes, which would not otherwise be recognizable by the inherent control system. The special modes might allow the amputee to engage in activities of daily living more naturally and comfortably, such as riding a bicycle, or sitting for long periods of time.

When prosthetic joint 101 is initialized, the default beginning is block 200 shown of FIG. 10, with the valve closed, which inspects the signals [load] and [moment] to detect heel strike at the beginning of the gait cycle. A positive [load] and a negative [moment] indicate weight on the heel. As the amputee transitions to loading response in block 201 of FIG. 10, both [load] and [moment] will become positive. At this time, the knee (i.e. prosthetic joint 101) could be bent, and a branch transition is seen between blocks 202 and 203 of FIG. 10. If the knee is bent, when the amputee reaches foot flat, the knee needs to allow extension. This knee extension is based on the aforementioned “stance extension” of the knee, when the force of the weight is acting on the toe, which generates a torque about the knee joint that tends to straighten, or extend, the knee. This block transition investigates [angle] to determine entering 202 or 203 of FIG. 10. The next block, 204 of FIG. 10, uses the [moment] and [load] signal again to determine if the amputee has loaded the unit fully by the toe. As [moment] will be greater than [load] when the toe is fully weighted, when the opposite is true, the microcontroller will know that weight is coming off the toe, and the user is initiating the next step. When [moment] is less than or equal to [load], the internal hydraulic valve will open to allow swing flexion, block 205 of FIG. 10.

Block 205 of FIG. 10 enables a prediction of the maximum desired heel rise, or knee [angle] based on the initial maximum of the knee angular velocity ([velocity]) signal. Gait speed tends to increase peak knee angle in proportion to foot fall temporal separation, otherwise known as cadence, or the time between the same gait event on a single side. Thus, ADL control system 306 can predict maximum desired knee angle without needing a single set point variable that might or might not be accurate to this step. This can be used to gently slow knee flexion at peak heel rise, as demonstrated in block 206 of FIG. 10. In the case of going to block 207 of FIG. 10, [angle] went greater than [peakAngle], and the valve was closed to stop the knee from bending father than desired on this step. In the case of traveling from block 206 to 208 of FIG. 10, [velocity] had a zero crossing before [peakAngle] was achieved, and no hydraulic means were needed to reverse knee flexion.

Block 208 of FIG. 10 allows free extension of the unit, until the transition at the last ˜8 to ˜10 degrees of terminal swing extension. The final 8 to 10 degrees of knee extension are damped so the unit does not impact the terminal stops of the knee joint with excess force, thus disturbing the amputee. This terminal extension damping can be seen in block 209 of FIG. 10, and continues until the knee reaches [angle] less than 0 and [velocity] greater than a negative 5, indicating full extension.

One important absence from this state machine is the dependence on time, or timers, in level ground walking. This means that an exemplary state machine is highly adaptive to different gait speeds, or changes in gait speed, without re-programming or changes to user settings, a significant enhancement over the previous two, simpler control systems.

Additionally, as shown in FIG. 3, there is a plurality of programmable modes (also known as sub-systems) within ADL control system 306. Mode 1, identified as 307, is a flat-ground walking mode allowing the amputee walk on flat-ground. Mode 2, identified as 308, is a foot-over-foot stairs mode allowing the amputee to walk up and down a set of stairs. Mode 3, identified as 309, is a cycling mode allowing the amputee to ride a bicycle. The amputee can switch between the modes through a user application. The modes have the added benefit of exiting back into the level ground walking mode (i.e. Flat-ground walking mode 307) without user input.

Cycling mode 309 allows the amputee to ride a bicycle. Even without an exemplary data trace, one skilled in the art can readily imagine that riding a bicycle would generate a [velocity] data stream that looks more or less like a sine wave. The alternating portions of the sine wave, a positive peak ([velocity]>50) and a negative peak ([velocity]<−50) form the basis for remaining in the state while cycling.

Also in the case of cycling mode 309, it is easy to see how an increase in safety comes from automatically going back into level ground walking (i.e. Flat-ground walking mode 307 of FIG. 3) as the user stops pedaling for some amount of time, in this example more than 3 minutes. A tired and perhaps forgetful amputee who has been on an exercise bike would appreciate the safety of the knee ready to support them when they get off and start walking automatically.

It is contemplated that there can be additional programmable modes that can be tailored to the amputee's needs. Mode N, identified as 310, represents this functionality. These additional programmable modes can be developed and programmed by master control unit 102. For example, if the amputee is a golfer, a golfing mode can be developed and programmed. Such golfing mode enables the amputee to stand in a semi-crouched position with a bent knee offering full resistance to bending (knee and hip flexion), but low resistance to rising (knee and hip extension). Alternatively, if the amputee wishes to run, then a running mode can be developed and programmed, which enables the amputee to achieve higher allowances on knee angular velocity and maximum knee flexion. In yet another example, a sitting mode enables the amputee to sit for long periods of time at a desk or some other office space task, while the knee is in a low resistance mode. This feature, which would be difficult or nearly impossible to detect in a standard walking state machine, allows the user to freely position the knee by hand, or with the contralateral foot, while at a desk. This allows for greater comfort and freedom of motion while no support is required of the knee. Again, this mode is automatically exited, back to the walking state machine, when load above a certain threshold is applied ([load]>20), which is indicative of standing up or desiring support from the unit.

The present invention is not limited to these three programmable control systems. It is contemplated that there can be more than, or less than, three programmable control systems depending on the needs of the amputee. Similarly, the functionalities typically associated with each control system can vary depending on the needs of the amputee. For example, if the amputee has previous experience with prosthetics, then, depending on a medical professional's assessment, such amputee may forego beginner control system 101. It is contemplated that the functionalities typically associated with each control system can be altered by the manufacturer with updates to the software or algorithm.

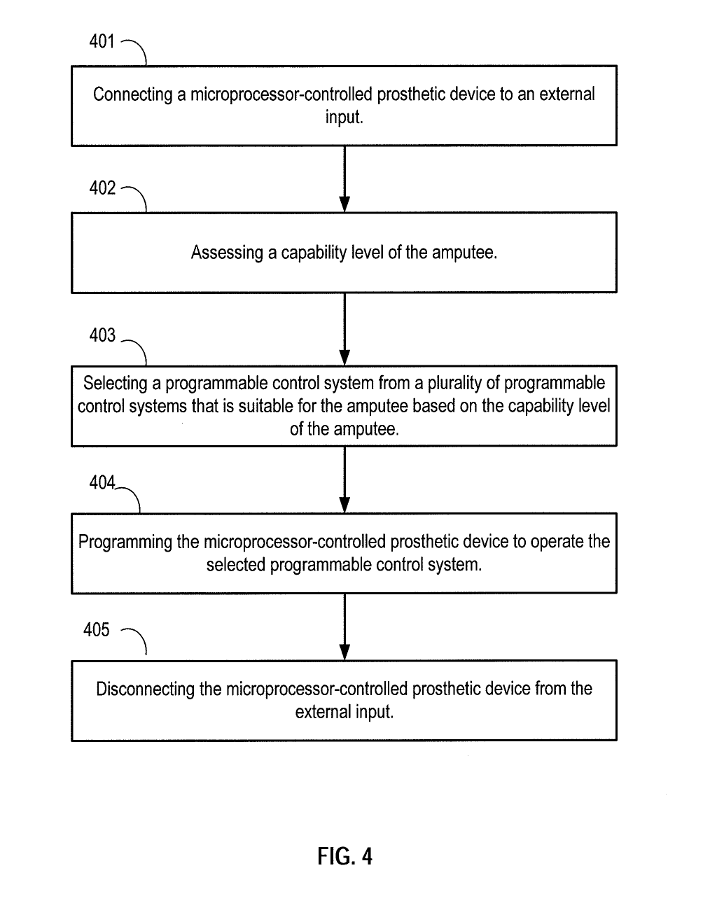

FIG. 4 is a flowchart depicting an exemplary embodiment of the present invention's method for a prosthetic device with multiple levels of functionality enabled through multiple control systems. At step 401, a microprocessor-control prosthetic device is connected to an external input. With reference to FIG. 1, the microprocessor-controlled prosthetic device can be prosthetic joint 101, and the external input can be master control unit 102. As shown in FIG. 1, prosthetic joint 101 can communicate with master control unit 102 via communication media 103.

At step 402, a capability level of the amputee is assessed for determining a programmable control system that is appropriate for the amputee. Typically, this step is performed by a medical professional who is qualified to make such assessment, such as a prosthetist. As shown in FIG. 3, the present invention introduces at least three controls system: beginner control system 301, intermediate control system 302, and advanced control system 303.

At step 403, a programmable control system is selected from a plurality of programmable control systems that is suitable for the amputee based on the capability level of the amputee. Like step 402, this step is performed by a qualified medical professional, such as a prosthetist. As shown in FIG. 3, beginner control system 301 is typically appropriate for a new amputee, intermediate control system 302 is typically appropriate for an amputee who has master beginner control system 301 or who is not yet suitable for advanced control system 303, and advanced control system 303 is typically appropriate for an amputee who has mastered intermediate control system 302.

At step 404, the microprocessor-controlled prosthetic device, such as prosthetic joint 101, is programmed to operate the selected programmable control system. As shown in FIG. 1, master control unit 102 programs prosthetic joint 101. These control systems can be programmed and modified by master control unit 102, then transferred to prosthetic joint 101 via communication media 103, and ultimately stored in machine-readable storage media 106.

At step 405, the microprocessor-controlled prosthetic device, such as prosthetic joint 101, is disconnected from the external input, such as master control unit 102. After the disconnection, controller 104 operates prosthetic joint 101 under the selected programmable control system. The programmable control systems are mutually exclusive such that prosthetic joint 101 only operates under the selected control system during the time period in which such system is selected.

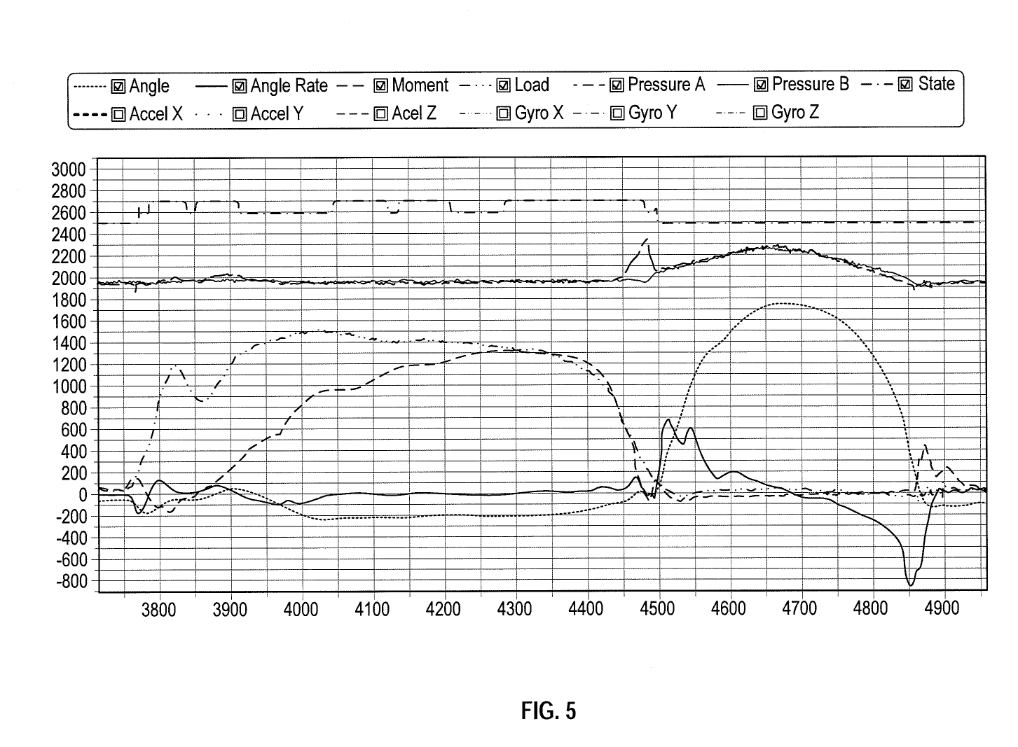

FIG. 5 is a representative data plot of sensor streams from an exemplary prosthetic knee, and included in the plot are unit state, knee angle, knee angular velocity, moment, load, pressure A, and pressure B. In the data stream, but not visualized to enhance clarity in the representative data plot, are 3 axis accelerometers and 3 axis gyros. As shown in the legend of the representative data plot, the 3 axis accelerators are accelerator x, accelerator y, and accelerator x. As shown in the legend of the representative data plot, the 3 axis gyros are gyro x, gyro y, and gyro z.

FIG. 6 is a descriptive algorithm path of a weight-activated stance control system, such as weight-activated stance control system 304.

FIG. 7 is a descriptive data stream of a weight-activated stance control system, such as weight-activated stance control system 304.

FIG. 8 is a descriptive algorithm path of a multiple gait speed, swing-and-stance control system, such as multiple gait speed, swing-and-stance control system 305.

FIG. 9 is a descriptive data steam of a multiple gait speed, swing-and-stance control system, such as multiple gait speed, swing-and-stance control system 305.

FIG. 10 is a descriptive algorithm path of an activities of daily living (ADL) control system, such as ADL control system 306.

FIG. 11 is a descriptive data stream path of an ADL control system for the level ground walking loop, such as ADL control system 306.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted.

What is claimed is:

1. A prosthetic device with multiple levels of functionality enabled through multiple control systems, said device comprising of:
a controller having a microprocessor;
a plurality of sensors;
an actuator;
a machine-readable storage medium for storing a plurality of programmable control systems, wherein each of the plurality of programmable control systems has at least one functionality different from each other; and
an interface configured to receive an external input, wherein the controller is configured to receive the external input from the interface.

2. The device of claim 1, wherein the external input programs the microprocessor-controlled prosthetic device to operate under one of the plurality of programmable control systems.

3. The device of claim 2, wherein the plurality of programmable control systems are mutually exclusive such that the microprocessor-controlled prosthetic device only operates under the selected control system during the time period in which such system is selected.

4. The device of claim 3, wherein the plurality of programmable control systems include at least a beginner control system, an intermediate control system, and an advanced control system.

5. The device of claim 4, wherein the intermediate control system has at least one functionality more than that of the beginner control system.

6. The device of claim 5, wherein the advanced control system has at least one functionality more than that of the intermediate control system.

7. The device of claim 6, wherein the beginner control system is a weight-activated stance control system.

8. The device of claim 7, wherein the intermediate control system is a swing-and-stance control system.

9. The device of claim 8, wherein the advanced control system is an activities of daily living control system.

10. The device of claim 6, wherein the advanced control system has a plurality of modes.

11. The device of claim 1, wherein the actuator has a hydraulic damping unit.

12. A control system for a prosthetic device with multiple levels of functionality enabled through multiple control systems, said system comprising of:
a master control unit configured to program a microprocessor-controlled prosthetic device;
the microprocessor-controlled prosthetic device having a controller, an actuator, a plurality of sensors, and a machine-readable storage medium;
the controller having a microprocessor;
the machine-readable storage medium for storing a plurality of programmable control systems, wherein each of the plurality of programmable control systems has at least one functionality different from each other; and
an interface configured to receive an external input, wherein the controller is configured to receive the external input from the interface.

13. The system of claim 12, wherein the actuator has a hydraulic damping unit.

14. A method for a prosthetic device with multiple levels of functionality enabled through multiple control systems, said method comprising the steps of:
connecting the microprocessor-controlled prosthetic device to an external input;
assessing a capability level of a user;
selecting a programmable control system from a plurality of programmable control systems that is suitable for the user based on the capability level of the user, wherein each of the plurality of programmable control systems has at least one functionality different from each other;
programming the microprocessor-controlled prosthetic device to operate the selected programmable control system, wherein the external input programs the microprocessor-controlled prosthetic device; and
disconnecting the microprocessor-controlled prosthetic device from the external input.

15. The method of claim 14, wherein the steps of assessing the capability level suitable of the user and selecting the programmable control system suitable for the user are performed by a qualified medical professional.

16. The method of claim 14, further comprising repeating the steps of claim 14 as the capability level of the user changes.

17. The method of claim 14, wherein the plurality of programmable control systems are mutually exclusive such that the microprocessor-controlled prosthetic device only operates under the selected control system during the time period in which such system is selected.

18. The method of claim 14, wherein the plurality of programmable control systems include at least a beginner control system, an intermediate control system, and an advanced control system.

19. The method of claim 14, wherein the plurality of programmable control systems include at least a weight-activated stance control system, a swing-and-stance control system, and an activities of daily living control system.

20. The method of claim 18, wherein the advanced control system has a plurality of modes.
